(19) United States
(12) Patent Application Publication
(10) Pub. No.: US 2019/0378598 A1
(43) Pub. Date: Dec. 12, 2019

(54) CENTRALIZED PHARMACY FOR VALIDATING PRESCRIPTION DRUGS

(71) Applicant: Nimble Rx, Inc., Menlo Park, CA (US)

(72) Inventors: Shaun Johnson, San Francisco, CA (US); Zoe Cohen, Stanford, CA (US); Talha Waqar, Palo Alto, CA (US)

(21) Appl. No.: 16/001,737

(22) Filed: Jun. 6, 2018

(51) Int. Cl. G16H 20/10; G16H 10/60; G06Q 40/08

(52) U.S. Cl. CPC G16H 20/10; G06Q 40/08; G16H 10/60

(57) ABSTRACT

A centralized pharmacy validates a drug prescribed by a physician for a patient to ensure that the prescribed drug is a safe and cost effective treatment for the patient. The centralized pharmacy accesses the medical history of the patient, drug information of the prescribed drug, the insurance health plan purchased by the patient, as well as drug abuse information. The centralized pharmacy performs the validation process using the accessed information. If the centralized pharmacy successfully validates the prescribed drug, the centralized pharmacy physically dispatches the prescribed drug to the patient. Alternatively, if the centralized pharmacy determines that the prescribed drug fails the validation process, the centralized pharmacy can identify an alternate drug and validates the alternate drug. Therefore, the centralized pharmacy can automatically replace the prescribed drug with a more appropriate, alternate drug and can dispatch the alternate drug to the patient.

### TECHNICAL FIELD

This disclosure generally relates to pharmacies, and more specifically to a centralized pharmacy that validates and dispatches prescribed drugs to patients.

### BACKGROUND

Patients typically visit a physician's office, are prescribed a drug by the physician, and subsequently visit a physical pharmacy location to fill the prescription. This process for filling a prescription is cumbersome as a patient must make separate visits to the physician's office and to the physical pharmacy. Furthermore, when a patient needs to refill a prescription, the patient may need to revisit the physician's office and/or revisit the pharmacy. Therefore, conventional means of obtaining prescription drugs is inefficient and burdensome on the patient.

Additionally, a patient must interact with multiple players (e.g., a physician, a pharmacist, and an insurance company) in order to successfully fill a prescription. This process can be susceptible to errors and may result in significant downstream problems. As one example, a pharmacist may incorrectly fill a prescription for the patient. As another example, a physician may accidentally prescribe a drug that may have adverse interactions with a drug that the patient is currently on. In another scenario, a patient with nefarious intentions may be able to take advantage of the disjointed nature of the drug prescription process. For example, a patient can approach various physicians to obtain prescriptions for drugs that can be abused, examples of which can include painkillers. In each of these examples and scenarios, conventional methods for providing a prescription drug to a patient can lead to poor outcomes for the patient.

### SUMMARY

A centralized pharmacy accesses information from various external systems to validate a drug prescribed by a physician for a patient. In particular, the centralized pharmacy validates the prescribed drug to ensure that the prescribed drug is a safe and cost effective treatment for the patient. To validate the prescribed drug, the centralized pharmacy may access information that includes one or more of the medical history of the patient, drug information of the prescribed drug, the insurance health plan purchased by the patient, and drug abuse information that is relevant for the prescribed drug. The centralized pharmacy analyzes the prescribed drug in view of the accessed information to validate the prescribed drug. In various embodiments, the centralized pharmacy can perform multiple validation processes for the prescribed drug, where each validation process refers to an analysis of the prescribed drug in view of accessed information from at least one external system. For example, the centralized pharmacy can perform a safety validation process for the prescribed drug using the drug information of the prescribed drug. As another example, the centralized pharmacy can perform a cost validation process for the prescribed drug using the patient's insurance information. If the prescribed drug is successfully validated, the centralized pharmacy can physically dispatch the prescribed drug to the patient. As an example, the centralized pharmacy can provide a notification indicating that the prescribed drug is to be physically mailed which subsequently causes the prescribed drug to be dispatched. Therefore, the patient can receive the prescribed drug at the patient's residence.

In some scenarios, the centralized pharmacy may determine that the drug prescribed by the physician fails the validation process. For example, the centralized pharmacy may determine that the prescribed drug may adversely interact with another drug taken by the patient. As another example, the patient may be abusing the prescribed drug but has nonetheless duped the physician into prescribing the drug. In these scenarios where the centralized pharmacy determines that the prescribed drug has failed the validation process, the centralized pharmacy can identify an alternate drug that serves as a replacement for the prescribed drug. The centralized pharmacy can further perform the validation process for the alternate drug to ensure that the alternate drug can be a safe and cost effective treatment for the patient. Thus, in these scenarios, the centralized pharmacy can modify the prescription to replace an inappropriately prescribed drug with a more appropriate, alternate drug.

The benefits of the implementation of centralized pharmacy as described herein are several fold. Specifically, the centralized pharmacy can directly communicate with devices operated by the physician and the patient while the patient is visiting the physician's office. The centralized pharmacy can perform the validation of the prescribed drug in real-time and therefore, the patient can confirm the delivery of the validated drug prior to leaving the physician's office. As such, the patient need not to physically visit a pharmacy location in order to fill a prescription and instead, can receive the validated drug at a residence address. In some scenarios, the drug can be delivered to the patient on the same day that the patient visits the physician's office. This process reduces the burden experienced by the patient when filling a prescription.

Secondly, by performing the validation process, the centralized pharmacy can ensure that the prescribed drug is appropriate for the patient. For example, the validation process can reduce the frequency of erroneous prescriptions that can arise due to human error (e.g., physician error or pharmacist error). Additionally, the validation process can ensure that the drug provided to the patient is affordable and/or covered by the patient's insurance policy. Furthermore, the validation process can identify possible scenarios where a patient is abusing a particular drug. Altogether, the centralized pharmacy ensures that the drug that is dispatched to the patient is safe and cost-effective for the patient.

### BRIEF DESCRIPTION OF THE DRAWINGS

The disclosed embodiments have advantages and features which will be more readily apparent from the detailed description, the appended claims, and the accompanying figures (or drawings). A brief introduction of the figures is below.

FIG. 1 depicts an overall system environment including a centralized pharmacy for validating prescription drugs, in accordance with an embodiment.

FIG. 2 depicts a block diagram architecture of a centralized pharmacy, in accordance with an embodiment.

FIG. 3 depicts an example flow diagram performed by a centralized pharmacy for validating and providing a drug to a patient, in accordance with an embodiment.

FIGS. 4A-4E each depicts an example user interface shown on a user kiosk, in accordance with an embodiment.

FIG. 5 depicts hardware components of an example computing device, in accordance with an embodiment.

The figures depict various embodiments for purposes of illustration only. One skilled in the art will readily recognize from the following discussion that alternative embodiments of the structures and methods illustrated herein may be employed without departing from the principles of the invention described herein.

Reference will now be made in detail to several embodiments, examples of which are illustrated in the accompanying figures. It is noted that wherever practicable similar or like reference numbers may be used in the figures and may indicate similar or like functionality. For example, a letter after a reference numeral, such as “patient personal information 475A,” indicates that the text refers specifically to the element having that particular reference numeral. A reference numeral in the text without a following letter, such as “patient personal information 475,” refers to any or all of the elements in the figures bearing that reference numeral (e.g. “patient personal information 475” in the text refers to reference numerals “patient personal information 475A” and/or “patient personal information 475B” in the figures).

### DETAILED DESCRIPTION

### Overall System Environment

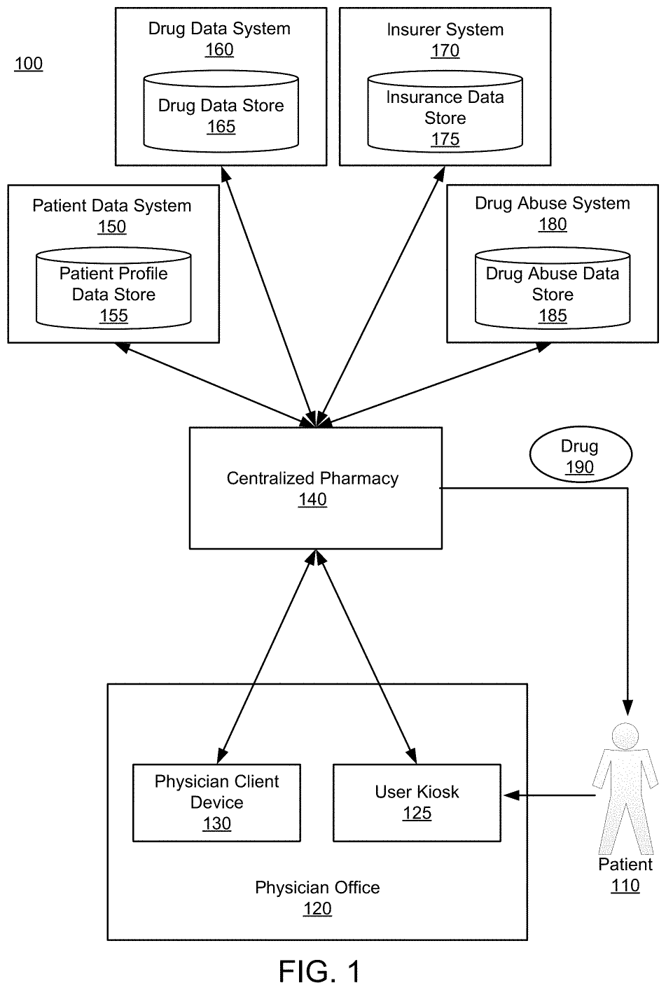

FIG. 1 depicts an overall system environment 100 that includes a centralized pharmacy 140 for validating prescription drugs, in accordance with an embodiment. The system environment 100 can include a centralized pharmacy 140, a patient data system 150, a drug data system 160, an insurer system 170, a drug abuse system 180, and a physician office 120 within which a physician client device 130 and a user kiosk 125 can operate. In various embodiments, the system environment 100 can include additional or fewer systems. For example, the system environment 100 can include multiple insurer systems 170 and/or multiple physician offices 120.

Each of the systems (e.g., centralized pharmacy 140 and systems 150, 160, 170, 180) as well as devices (e.g., physician client device 130 and user kiosk 125) are configured to communicate with one another through a network. Such a network may be any wired or wireless local area network (LAN) and/or wide area network (WAN), such as an intranet, an extranet, or the Internet and can use standard communication technologies and/or protocols. In various embodiments, each of the patient data system 150, drug data system 160, insurer system 170, drug abuse system 180, physician client device 130, and user kiosk 125 communicate with the centralized pharmacy 140 within the system environment 100. In other words, the centralized pharmacy 140 acts as the intermediary between the systems and devices. In other embodiments, some of the patient data system 150, drug data system 160, insurer system 170, drug abuse system 180, physician client device 130, and user kiosk 125 can communicate directly with one another. For example, although not explicitly shown in FIG. 1, the patient data system 150 can directly provide patient information to a physician client device 130 and/or to the user kiosk 125 without having to first provide the patient information to the centralized pharmacy 140.

Generally, the centralized pharmacy 140 receives a prescription that identifies a prescription drug. The prescription may be provided by a physician (e.g., through the physician client device 130). The centralized pharmacy 140 validates the prescription drug by using information accessed from various databases across the patient data system 150, drug data system 160, insurer system 170, and drug abuse system 180. As used herein, validating a drug refers to ensuring that the drug satisfies one or more criteria, for instance, criteria related to safety, cost, and/or drug abuse.

If the centralized pharmacy 140 validates the prescribed drug, the centralized pharmacy 140 can provide information identifying the validated drug to the user kiosk 125. Therefore, the patient 110 for whom the drug is prescribed for can view the details of the validated prescribed drug and can request for the prescription to be filled. The centralized pharmacy 140 can dispatch the prescribed drug to the patient 110 through physical means, such as through mail or parcel delivery.

In some scenarios, the centralized pharmacy 140 may determine that the prescribed drug fails the validation process. Thus, the centralized pharmacy 140 can replace the prescribed drug with an alternate drug that satisfies the validation process. As an example, the centralized pharmacy 140 may determine that the prescribed drug is not covered by the patient's insurance plan, but an alternate drug that is safe and therapeutically effective is covered by the patient's insurance plan. As such, the centralized pharmacy 140 can, in real-time, update the prescription to include the alternate drug in lieu of the prescribed drug, and can provide details of the alternate drug to the user kiosk 125 for viewing by the patient 110. Upon receiving a confirmation from the user kiosk 125, the centralized pharmacy 140 can dispatch the alternate drug for the patient through physical means (e.g., through mail or parcel delivery).

In some embodiments, upon determining that the prescribed drug fails the validation process, the centralized pharmacy 140 may provide a notification to the physician client device 130 and/or the user kiosk 125 that indicates that the prescribed drug failed the validation process. Thus, the centralized pharmacy 140 notifies the physician and/or the patient 110 that the prescribed drug is not appropriate for the patient 110. In some embodiments, in addition to providing a notification that the prescribed drug failed the validation process, the centralized pharmacy 140 may provide identification of alternate drugs to the physician client device 130 and/or the user kiosk 125. Here, a patient 110 that accesses the user kiosk 125 may provide approval of an alternate drug and therefore, the centralized pharmacy 140 can provide the alternate drug to the patient 110. In some embodiments, the centralized pharmacy 140 may seek the physician's approval for the alternate drug prior to providing the alternate drug to the patient 110.

The physician office 120 refers to the location where a patient 110 visits to seek a physician. As shown in FIG. 1, the physician office 120 can include a physician client device 130 and a user kiosk 125. In other embodiments, the physician office 120 can include additional devices such as multiple physician client devices 130 to be accessed by multiple physicians of the physician office and/or multiple user kiosks 125 to be accessed by multiple patients 110 that visit the physician office 120.

The physician client device 130 and the user kiosk 125 may each be an electronic device such as a personal computer (PC), a desktop computer, a laptop computer, a notebook, a tablet PC executing an operating system, for example, a Microsoft Windows-compatible operating system (OS), Apple OS X, and/or a Linux distribution. In another embodiment, each of the physician client device 130 and the user kiosk 125 can be any device having computer functionality, such as a personal digital assistant (PDA), mobile telephone, smartphone, etc. Each of the physician client device 130 and user kiosk 125 may execute instructions (e.g., computer code) stored on a computer-readable storage medium. Each of the physician client device 130 and user kiosk 125 may each include one or more executable applications, such as a web browser, to interact with services and/or content provided by the centralized pharmacy 140. In another scenario, the executable application may be a particular application designed by the centralized pharmacy 140 and locally installed on either the physician client device 130 or user kiosk 125. As a particular scenario, a physician can access the physician client device 130 and may register with the centralized pharmacy 140 using a user identifier and password. As another scenario, a patient 110 can access the user kiosk 125 and may register with the centralized pharmacy 140 using a different user identifier and different password.

Referring to the physician client device 130, it enables a physician to prepare drug prescriptions that, when submitted by the physician, transmits the drug prescriptions to the centralized pharmacy 140. In various embodiments, the physician client device 130 may include software or programs that facilitates the prescription process for a physician who accesses the physician client device 130. For example, the physician client device 130 can include a database of available prescription drugs such that the physician can, with ease, identify appropriate prescription drugs that can be prescribed for a patient 110. Therefore, the physician can search the database to identify a drug and electronically prescribe the drug using the physician client device 130.

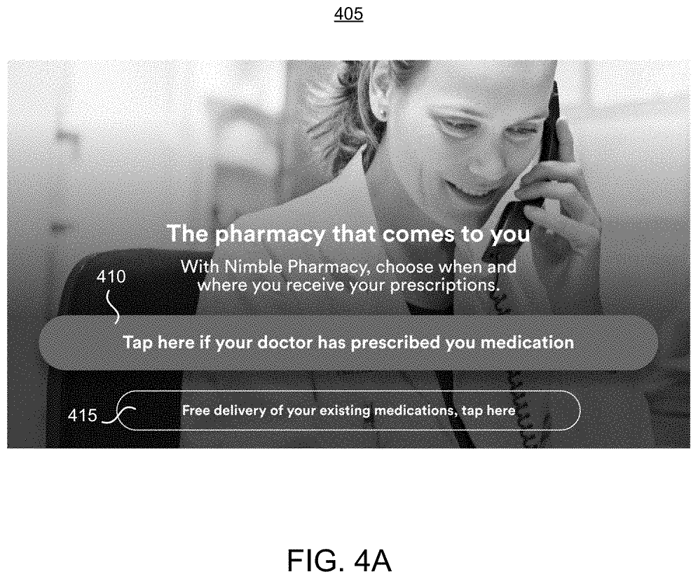

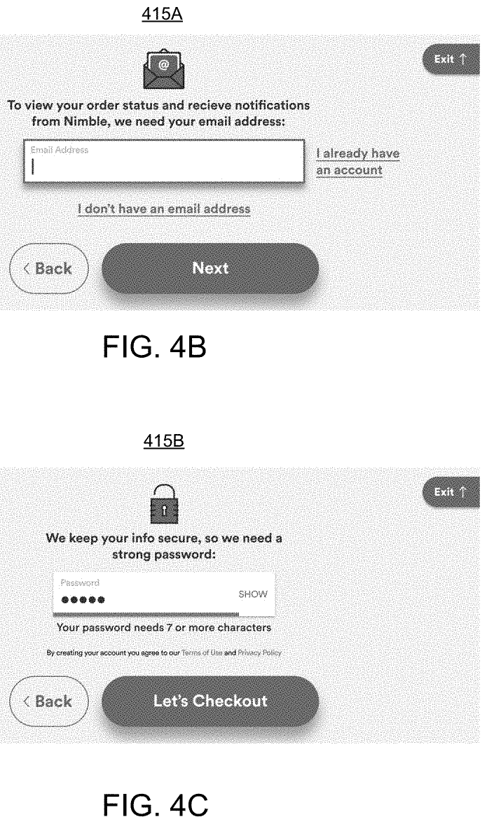

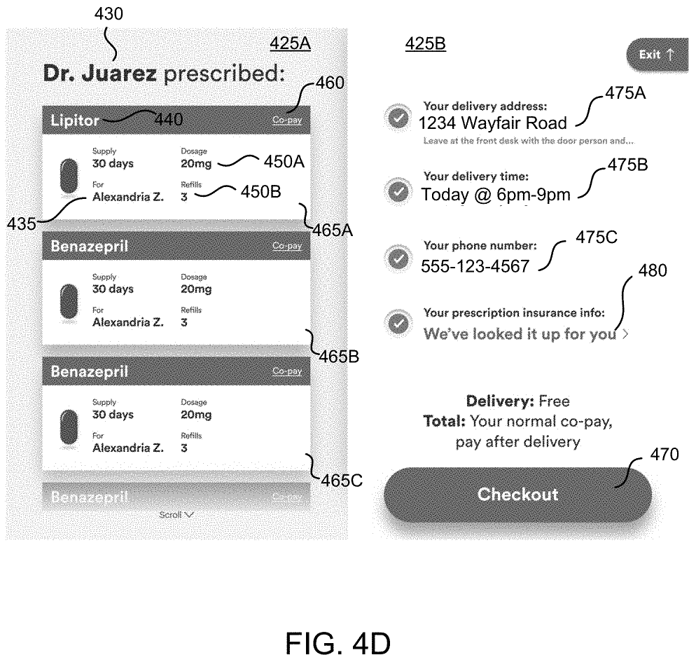

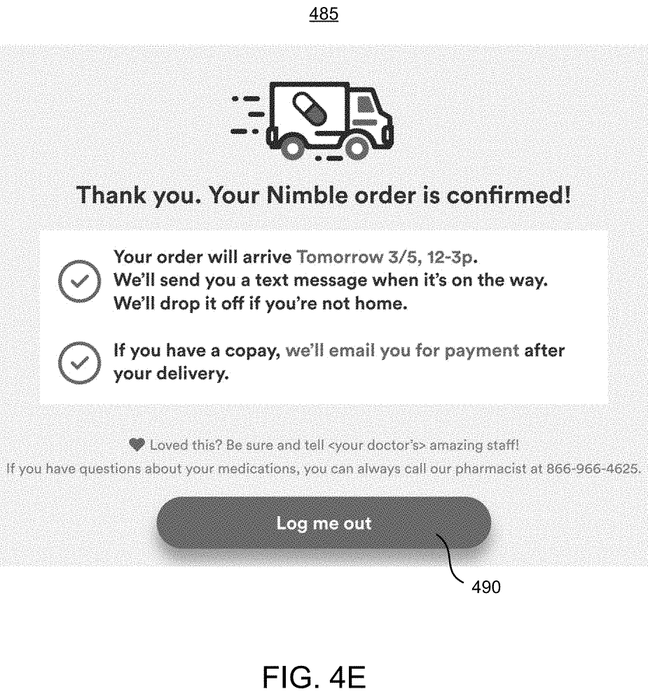

The user kiosk 125 may be an apparatus located in an area that is generally accessible to patients 110. As an example, the user kiosk 125 can be located within the receptionist or waiting area of the physician office 120. In various embodiments, the user kiosk 125 may include a screen for displaying information to the patient 110. Here, the display screen can provide a user interface that displays information such as an identification of a prescribed drug, information relevant to the prescribed drug (e.g., dose, supply, and the like), personal information of the patient 110, identifying information of the physician, and the like. Further description in relation to example user interfaces of the user kiosk 125 is described below in relation to FIGS. 4A-4C.

Generally, a patient 110 that accesses the user kiosk 125 can view one or more drugs can approve the delivery of the drugs to a delivery address. In various embodiments, the one or more drugs shown to the patient on the user kiosk 125 have successfully passed the validation process performed by the centralized pharmacy 140. Through the user kiosk 125, a patient 110 can directly fill the prescribed drugs on the spot at the physician office 120 without having to visit a separate pharmacy establishment. In one embodiment, the patient 110 can provide personal information such as a patient's mailing address. Therefore, the patient 110 can receive the prescribed drugs through the mail at his/her mailing address subsequent to filling the prescription at the user kiosk 125 within the physician office 120. In various embodiments, the patient 110 can receive prescribed drugs through the mail at a specified mailing address within a threshold amount of time (e.g., 1, 2, 3, 4, 5, 6, 7, 8, 9, 10 hours, or within a day) after filling the prescription at the user kiosk 125.

The patient data system 150 represents a system that manages patient information. Patient information can be organized as electronic health records. The patient data system 150 can access and provide patient information to the centralized pharmacy 140. In some embodiments, the patient data system 150 can provide patient information directly to the physician office 120 such that a physician can obtain relevant patient information when the patient visits the physician office 120. Examples of the patient data system 150 can be CARE EVERYWHERE of Epic Systems Corporation, CERNER MILLENIUM of Cerner Corporation, EXPANSE of Medical Information Technology, and SUNRISE of Allscripts Healthcare Solutions, Inc.

The patient data system 150 may include a patient profile data store 155 which stores individualized patient profiles. Each patient profile includes patient information for a particular patient 110. In one embodiment, each patient profile can be identifiable based on a patient identifier stored in the patient profile. Examples of a patient identifier can be a uniquely assigned identifier for a patient, a name of the patient, or patient contact information such as an email address of the patient. Therefore, the patient data system 150 can identify the appropriate patient profile if provided a patient identifier.

Patient information stored in a patient profile can be initially provided by the patient 110 at a physician office 120 and subsequently transmitted to the patient data system 150. Each patient profile can be updated each time new patient information is received (e.g., each time the patient 110 visits the physician office 120). Therefore, the patient profile for a patient can include tracked patient information across years of physician visits. Examples of patient information stored in a patient profile data store 155 can include personal information of the patient (e.g., patient's name, physical address, contact information, age, gender, and the like), medical history of the patient (e.g., prior indications, family medical history, prior or current drugs that have/are being taken, prior or current treatments, patient allergies, prior prescriptions that have been filled), and physician information (e.g., name of visited physician, physician's notes from a prior visit).

The drug data system 160 stores drug information for drugs, such as drugs that can be prescribed by a physician for a patient 110. The drug data system 160 includes a drug data store 165. In one embodiment, drug information in the drug data store 165 can be organized on a per-drug basis. For example, for each drug, the drug data store 165 can organize drug information for the drug in subcategories including 1) drug treatment information, 2) risk information associated with the drug, and 3) relevant additional drug information. Drug information can be organized based on an identifier of the drug, examples of which include a chemical name of the drug, a brand name of the drug, or an abbreviation of the drug. Therefore, the drug data system 160 can identify the appropriate drug information stored in the drug data store 165 if provided the identifier of the drug.

The drug treatment information refers to how the drug is used to treat a patient. For example, drug treatment information can include the Food and Drug Administration (FDA) approved method of administering the drug to the patient (e.g., dose, dose frequency, or type of administration such as oral consumption or intravenous injection). As other examples, drug treatment information can include the drug's mode of action, drug target, target pathway, drug molecular structure, and the like.

The risk information associated with the drug refers to possible adverse interactions that may arise if the patient were to take the drug. As an example, the risk information can include one or more incompatible drugs that, if taken with the drug, are known to cause adverse interactions in the patient. In various embodiments, the risk information associated with the drug can be partitioned into different risk categories, examples of which include high risk, moderate risk, low risk, and no risk categories. Thus, each of the one or more incompatible drugs included in the risk information can be categorized in a risk category.

The relevant additional drug information refers to information pertaining each of one or more additional drugs that are relevant to the drug. In some embodiments, each additional drug relevant to the drug can be an additional drug that has the same therapeutic effect as the drug and therefore, can be potentially taken by a patient in lieu of the drug. As one example, if the drug is a brand name drug, the one or more additional drugs can be a generic drug. As another example, the additional drug can have a different mode of action that results in a similar therapeutic effect. In some embodiments, each additional drug is commonly prescribed by physicians in addition to the drug. For example, if the drug is part of a treatment regimen, where the treatment regimen further includes secondary drugs that are commonly taken by the patient in addition to the drug, the secondary drugs can be additional drugs that are identified in the relevant additional drug information.

In some embodiments, the drug data system 160 can be a publicly available database that stores drug information. Examples of the drug data system 160 can be the DRUGBANK, the Human Metabolome Database (HMDB), the Toxin and Toxin Target Database (T3DB), or the Small Molecule Pathway Database (SMPDB).

The insurance system 170 manages insurance plans of patients 110. Examples of an insurer system 170 can be any one of UnitedHealth Group, Inc., Aetna Inc., Cigna, and Anthem, Inc. The insurance system 170 can include an insurance data store 175 that stores information of individual patient insurance plans. In one embodiment, each insurance plan that belongs to a patient can be identifiable based on a patient identifier stored in association with the insurance plan. Examples of a patient identifier can be a uniquely assigned identifier, a name of the patient, or patient contact information such as an email address of the patient. Therefore, the insurance system 170 can identify and retrieve the appropriate insurance plan if provided a patient identifier.

Each insurance plan may specify a type of insurance plan that the patient 110 has purchased. Examples of a type of insurance plan can be an exclusive provider organization (EPO), a point of service (POS), a health maintenance organization (HMO), or a preferred provider organization (PPO) insurance plan. Each insurance plan can include identifications particular drugs and/or treatments, whether each drug and/or treatment are each covered or not covered by the insurance plan, the quantity or dose of the drug or treatment that is covered by the insurance plan, and payment costs for each drug or treatment depending on whether they are covered or not covered by the insurance plan. For example, an insurance plan can include a list (e.g., a list of covered drugs) that includes identifiers of drugs that are covered by the insurance plan and a cost for each of the drugs that are covered by the insurance plan. Additionally, the insurance plan can include a list (e.g., a list of not covered drugs) of identifiers of other drugs that are not covered by the insurance plan as well as the cost for each of these other drugs that are not covered by the insurance plan. As a specific example, an insurance plan may be a PPO insurance plan that specifies that the drug LIPITOR is covered by the insurance plan and therefore, the patient 110 is responsible for only a copayment cost.

An insurance plan can include further restrictions set by the insurer system 170. For example, an insurance plan can specify restrictions such as quantity limits for certain drugs (e.g., dose limits or frequency of refill limits) or a need for prior authorization for certain drugs (e.g., authorization must be obtained from a physician or from an insurer). As described above, the insurance plan can include a list of covered drugs and therefore, the list can further include restrictions for each drug in the list. As an example, the list of covered drugs can specify, for each drug, whether authorization is required prior to providing the drug to the patient.

In various embodiments, the insurance data store 175 can further store discount information such as coupons, offers, and promotions that can be applied for particular drugs and treatments. Such discount information can identify a drug or treatment that the discount is applied for, a duration during which the discount is valid for, and specific insurance plans (e.g., EPO, POS, HMO, or PPO) that the discount is valid for.

The drug abuse system 180 manages drug abuse information that can be used to identify whether a patient is potentially abusing the use of drugs. In various embodiments, examples of the drug abuse system 180 can be the National Institute on Drug Abuse (NIDA), Prescription Drug Abuse Policy System (PDAPS), or the National Drug Early Warning System (NDEWS).

The drug abuse system 180 can include a drug abuse data store 185 that that identifies commonly abused drugs. Commonly abused drugs can be categorized in categories such as stimulants, depressants, hallucinogens, dissociatives, opioids, inhalants, and cannabis. For each commonly abused drug, the drug abuse data store 185 may include drug abuse information. The drug abuse data store 185 may organize drug abuse information according to each commonly abused drug's identifier (e.g., name of abused drug, abbreviation of abused drug, and the like). Drug abuse information for each drug can describe common observable symptoms (e.g., poor memory, ragged appearance, slurred speech, lethargy, and the like) of patients that abuse the drug. Additionally, drug abuse information can identify drug abuse patient actions that can be indicative of abuse of the drug. Examples of drug abuse patient actions can be a frequency of a refill rate of an abusable drug or the total number of different physicians that a patient has visited over a particular time span to obtain prescriptions for an abusable drug.

### Centralized Pharmacy

The centralized pharmacy 140 interfaces with each of the systems 150, 160, 170, 180 and devices 125 and 130 in physician offices 120 to validate and provide drugs to patients 110. Generally, the centralized pharmacy 140 receives a prescription of a drug that is prescribed by a physician for a patient 110. The centralized pharmacy 140 accesses the data stores 155, 165, 175, and 185 of the different systems to perform one or more validations of a drug that is prescribed by a physician for a patient 110. By performing the validation steps, the centralized pharmacy 140 can ensure that the prescribed drug is a safe and cost effective treatment for the patient. If the prescribed drug fails a validation step, the centralized pharmacy 140 can identify an alternate drug for the patient and can replace the prescribed drug in the prescription with the alternate drug. Thus, the centralized pharmacy 140 can provide a drug 190 that has passed the validation steps to the patient 110. As an example, the centralized pharmacy 140 can provide an indication that the drug 190 has passed the validation steps, thereby causing the drug 190 to be physically dispatched (e.g., through the mail) to the patient 110.

In some embodiments, the centralized pharmacy 140 can be embodied as a cloud server or rack server. In other words, the functions and algorithms performed by the centralized pharmacy 140 can be distributed across multiple processors and/or electronic devices. In some embodiments, the processors are located in a single geographic location (e.g., within a single location of the centralized pharmacy 140). In other embodiments, the processors may be distributed across a number of geographic locations.

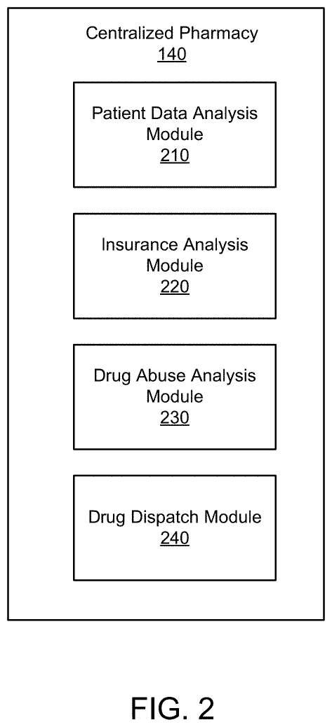

Referring to FIG. 2, it depicts a block diagram architecture of a centralized pharmacy 140, in accordance with an embodiment. The centralized pharmacy 140 can include a patient data analysis module 210, an insurance analysis module 220, a drug abuse analysis module 230, and a drug dispatch module 240. Generally, each of the modules 210, 220, and 230 performs at least one validation process for determining whether a drug is appropriate for a patient 110. For example, the patient data analysis module 210 may perform a safety validation for a drug, the insurance analysis module 220 may perform a cost validation for a drug, and the drug abuse analysis module 230 may perform an abuse validation for a drug. In other embodiments, the centralized pharmacy 140 can include additional modules for performing additional validation steps. As used hereafter, each of the data analysis module 210, insurance analysis module 220, and drug abuse analysis module 230 will be described in relation to validating a “target drug.” In various embodiments, the target drug can be the drug prescribed by the physician and therefore, a module validates the prescribed drug. In some embodiments, the target drug can be an alternate drug that has been identified after the prescribed drug has failed a validation process. Therefore, a module can validate an alternate drug.

Referring first to the patient data analysis module 210, it analyzes a target drug in view of patient information accessed from the patient profile data store 155 of the patient data system 150. Generally, the patient data analysis module 210 performs a safety validation of the target drug to ensure that the target drug can be safely taken by the patient by using a combination of patient information and drug information. In various embodiments, if the patient data analysis module 210 determines that a target drug fails the safety validation process, the patient data analysis module 210 may provide a notification to either the physician client device 130 and/or the user kiosk 125 to notify the physician and/or the patient 110 of the failed safety validation. In some embodiments, if the patient data analysis module 210 determines that a target drug fails the safety validation process, the patient data analysis module 210 may identify one or more alternative drugs and validate each of the one or more alternative drugs based on the patient information and drug information of each of the one or more alternative drugs. The patient data analysis module 210 may provide a list of one or more validated drugs to a subsequent module of the centralized pharmacy 140 for subsequent validation of each of the drugs on the list.

The patient data analysis module 210 obtains patient information of the patient from the patient profile data store 155 by sending a patient information request to the patient data system 150. In various embodiments, the patient information request includes an identifier of the patient, examples of which include a unique identifier of the patient, the patient's name, or patient contact information such as the email address of the patient. In various embodiments, the patient information request may be encrypted to protect the privacy of the patient. The patient data analysis module 210 receives the patient information of the patient that is accessed from the patient profile data store 155. The patient data analysis module 210 further obtains drug information for the target drug by sending a drug information request to the drug data system 160. In various embodiments, the drug information request includes an identifier of the target drug, such as a name of the target drug (e.g., brand name or chemical name). In return, the drug data system 160 returns the drug information of the target drug that is accessed from the drug data store 165.

The patient data analysis module 210 compares the patient information to the drug information to determine whether the patient can safely take the target drug. In one embodiment, the patient data analysis module 210 compares the medical history of the patient that is included in the patient information to each subcategory of the drug information (e.g., drug treatment information, risk information, and relevant additional drug information). The patient data analysis module 210 may compare the medical history to each subcategory of the drug information in parallel. In various embodiments, the patient data analysis module 210 determines that the target drug passes the safety validation process if each of the comparisons yields a satisfactory result.

By comparing the medical history of the patient to drug treatment information, the patient data analysis module 210 determines whether the patient has been compliantly following prior medical advice. Specifically, if the patient is currently refilling a prescription for a target drug, the patient data analysis module 210 determines whether the refill of the drug prescription is reasonably within certain limits. In various embodiments, the certain limits refers to a threshold amount of time (e.g., number of days or number of months) that differs from the expected amount of time that a supply of a target drug is expected to last. For example, the drug treatment information may specify that a target drug is to be orally ingested once per day. Therefore, if the medical history of the patient indicates that a thirty day supply of the drug was prescribed and filled a week ago, the patient data analysis module 210 may determine that the refill of the drug prescription exceeds expected limits because it is unlikely that the patient requires a refill so soon. Conversely, if the medical history of the patient indicates that a thirty day supply of the drug was prescribed and filled six months ago, the patient data analysis module 210 may similarly determine that the refill of the drug prescription exceeds expected limits. Here, the patient data analysis module 210 may determine that the prescribed drug fails the safety validation process based on the patient information (medical history of the patient) and the drug information (e.g., drug treatment information).

By comparing the medical history of the patient to risk information associated with the drug, the patient data analysis module 210 can determine whether the target drug, if taken by the patient, can lead to adverse interactions with prior drugs and/or treatments that are identified in the medical history of the patient. Specifically, the patient data analysis module 210 compares drugs identified in the medical history of the patient to drugs that are included in the risk information of the target drug and determines whether the comparison identifies a possible risk. In one embodiment, if the comparison yields a possible risk between a drug in the medical history and a drug in the risk information of the target drug, the patient data analysis module 210 can deem that the target drug has failed the safety validation process. Here, an identified risk between two drugs can refer to an alignment between drug identifications. For example, a possible risk between two drugs may be an alignment between a brand name for a drug (e.g., LIPITOR) in the patient's medical history and the scientific name of the same drug (e.g., atorvastatin) in the risk information of the target drug. As another example, an alignment can refer to the same mode of action between the two drugs. Therefore, a first drug in the patient's medical history that has the same mode of action as a second drug that is included in the risk information of the target drug, even if they are differently named, can be identified as a possible risk. In various embodiments, an identified possible risk between two drugs is further dependent on the doses of the two drugs. For example, if the patient's medical history identifies that the patient has been taking a first drug at a particular dose, then a target drug is a possible risk if the first drug appears in the risk information of the target drug and the particular dose of the first drug is within a threshold amount of a dose specified in the risk information of the target drug. In some embodiments, the threshold amount may be a range (e.g., ±10% of the dose specified in the risk information).

In some embodiments, the patient data analysis module 210 compares the drugs identified in the medical history of the patient to drugs that are included in the risk information of the target drug and calculates a risk score that represents a severity of a possible adverse interaction that may occur if the patient were to take the target drug. The patient data analysis module 210 may deem the target drug as failing the safety validation process if the risk score is above a threshold value. Conversely, the patient data analysis module 210 may deem the target drug as passing the safety validation process if the risk score is below the threshold value. In various embodiments, the patient data analysis module 210 may determine a risk score based on the risk category included in the risk information that a drug is categorized within. For example, if a first drug is categorized in a high risk category in the risk information of a target drug, the patient data analysis module 210 may assign a higher risk score to the first drug in comparison to a risk score assigned to a second drug that is included in a moderate risk category in the risk information of the target drug.

By comparing the medical history of the patient to relevant additional drug information, the patient data analysis module 210 can determine whether a different drug should be prescribed either in lieu of or in addition to the target drug. The patient data analysis module 210 can identify one or more additional drugs included in the relevant additional drug information. Here, each of the one or more additional drugs can have a different mode of action than the target drug and therefore, may need to be re-validated to ensure that each of the one or more additional drugs can be safely taken by the patient. Therefore, for each additional drug, the patient data analysis module 210 may additionally perform the safety validation process to validate each additional drug. Specifically, the patient data analysis module 210 compares the medical history of the patient that is included in the patient information to the drug treatment information of the additional drug and to the risk information of the additional drug.

In various embodiments, the patient data analysis module 210 generates a list of drugs that the patient data analysis module 210 has successfully validated. For example, in one embodiment, the list of drugs can include the initial drug prescribed by the physician and one or more additional drugs that are typically included in the treatment regimen that includes the initial drug. In another embodiments, the list of drugs can include an alternate drug that serves as an alternative to the initial drug prescribed by the physician. Here, the list of drugs may not include the initial drug prescribed by the physician because the patient data analysis module 210 may have deemed the initial drug as failing the safety validation process. The patient data analysis module 210 can provide the list of drugs to a subsequent module for a subsequent validation of each drug included in the list.

The insurance analysis module 220 analyzes a target drug, such as a drug included in the list provided by the patient data analysis module 210, in view of an insurance plan accessed from the insurance data store 175 of the insurance system 170. Generally, the insurance analysis module 220 performs a cost validation of the target drug to ensure that the target drug is a cost effective drug for the patient. In various embodiments, if the insurance analysis module 220 determines that a target drug fails the cost validation process, the insurance analysis module 220 may provide a notification to either the physician client device 130 and/or the user kiosk 125 to notify the physician and/or the patient 110 of the failed cost validation. In some embodiments, if the insurance analysis module 220 determines that a target drug fails the cost validation process, the insurance analysis module 220 may identify an alternate drug and validates the alternate drug based on the insurance plan accessed from the insurance data store 175 of the insurance system 170. The insurance analysis module 220 may provide an identification of the validated drug to a subsequent module, such as the drug abuse analysis module 230, for subsequent validation.

The insurance analysis module 220 obtains an insurance plan associated with the patient from the insurance data store 175 by sending an insurance plan request to the insurance system 170. In various embodiments, the insurance plan request includes an identifier of the patient, examples of which include a unique identifier of the patient, the patient's name, or patient contact information such as the email address of the patient. In return, the insurance system 170 provides the patient's insurance plan that is accessed from the insurance data store 175.

The insurance analysis module 220 analyzes the patient's insurance plan to determine whether a target drug is cost-effective for the patient. In various embodiments, the insurance analysis module 220 analyzes the patient's insurance plan for each target drug included in the list provided by the patient data analysis module 210. The insurance analysis module 220 identifies the type of the insurance plan (e.g., EPO, POS, HMO, PPO) that the patient has purchased and determines whether the target drug is covered by the particular type of insurance plan and whether the target drug is subject to any restrictions of the type of insurance plan.

As described above, an insurance plan can include a list of drugs that are covered by the insurance plan and a list of drugs that are not covered by the insurance plan. Therefore, for each target drug, the insurance analysis module 220 parses the list of covered drugs to determine whether the target drug is covered by the insurance plan. For example, the insurance analysis module 220 parses the drug identifiers in the list of covered drugs to identify a match between an identifier of the target drug and an identifier of the target drug in one of the lists. In various embodiments, if the insurance analysis module 220 determines that the target drug is not covered by the insurance plan (e.g., not included in the list of covered drugs), the insurance analysis module 220 can deem the target drug as failing the cost validation process in response to that determination.

In some embodiments, if the insurance analysis module 220 determines that the target drug is covered by the insurance plan, the insurance analysis module 220 determines whether the target drug satisfies restrictions included in the insurance plan, if any, that have been previously set on the target drug. For example, by parsing the list of covered drugs, the insurance analysis module 220 determines whether the particular dose of the target drug (e.g., a dose of the target drug prescribed by a physician) is covered by the insurance plan. As another example, by parsing the list of covered drugs, the insurance analysis module 220 determines whether any required authorization (e.g., authorization from a physician or authorization from the insurer system 170) is needed before the target drug can be provided to the patient. If authorization is required, the insurance analysis module 220 can send a request to the appropriate party for authorization (e.g., to the physician client device 130 if physician authorization is required or to the insurer system 170 if authorization from the insurer system 170 is required).

In various embodiments, if the insurance analysis module 220 determines that the target drug is covered by the patient's insurance plan and passes restrictions specified by the insurance plan, the insurance analysis module 220 can deem the target drug as passing the cost validation process. In contrast, if the insurance analysis module 220 determines that the target drug is not covered by the patient's insurance plan or does not pass a restriction of the insurance plan, the insurance analysis module 220 can deem the target drug as failing the cost validation process.

If the insurance analysis module 220 determines that a target drug fails the cost validation process, the insurance analysis module 220 can identify an alternate drug that is more cost-effective (e.g., more cost-effective in comparison to the target drug). In various embodiments, the insurance analysis module 220 accesses relevant additional drug information from the drug data store 165 that identifies one or more additional drugs included in the relevant additional drug information. Thus, the insurance analysis module 220 can perform the cost validation process on each of the additional drugs. Here, if the insurance analysis module 220 identifies that an additional drug passes the cost validation process, then the insurance analysis module 220 has identified an alternate drug that is covered by the patient's insurance plan and satisfies any restrictions placed on the alternate drug.

In various embodiments, the insurance analysis module 220 may notify the patient data analysis module 210 that a target drug, which had previously passed the safety validation process performed by the patient data analysis module 210, has failed the cost validation process and that an alternate drug has been identified. Therefore, the insurance analysis module 220 can enable the patient data analysis module 210 to perform the safety validation process on the alternate drug, if the patient data analysis module 210 has not done so already.

In some embodiments, the insurance analysis module 220 can further apply a discount using discount information accessed from the insurance data store 175 to reduce the cost of the target drug for the patient. For example, the insurer system 170 may be operating an ongoing promotion for the target drug. Therefore, the insurance analysis module 220 can apply the discount, thereby reducing the cost burden on the patient.

The drug abuse analysis module 230 performs an abuse validation process for a target drug, such as a drug validated by both the patient data analysis module 210 and the insurance analysis module 220, to ensure that the patient is not abusing the target drug. In various embodiments, the drug abuse analysis module 230 performs the abuse validation process using drug abuse information accessed from the drug abuse data store 185 of the drug abuse system 180. In some embodiments, the drug abuse analysis module 230 additionally uses patient information accessed from the patient profile data store 155 of the patient data system 150 to perform the abuse validation process for the target drug. In various embodiments, if the drug abuse analysis module 230 determines that a target drug fails the abuse validation process, the drug abuse analysis module 230 may provide a notification to the physician client device 130 to notify the physician of the failed safety validation. In various embodiments, the drug abuse analysis module 230 will not allow the target drug to be provided to the patient 110 if the target drug has not passed the abuse validation process. In other words, in these embodiments, the abuse validation process must be passed in order for the target drug to be provided to the patient 110.

The drug abuse analysis module 230 obtains drug abuse information of the target drug from the drug abuse data store 185 by sending a drug abuse request to the drug abuse system 180. In various embodiments, the drug abuse request includes an identifier of the target drug, examples of which include a name of the drug or an abbreviation of the drug. If the target drug is an abusable drug with information that is stored in the drug abuse data store 185, the drug abuse analysis module 230 receives drug abuse information for the target drug from the drug abuse system 180. Alternatively, if the target drug is not an abusable drug, the drug abuse system 180 may not have drug abuse information for the target drug. Therefore, the drug abuse analysis module 230 may receive a notification that indicates that the target drug is not likely to be abused by the patient 110.

If the drug abuse analysis module 230 receives drug abuse information for the target drug, the drug abuse analysis module 230 can analyze the drug abuse information to determine whether the patient is likely abusing the target drug. In one embodiment, the drug abuse analysis module 230 may analyze the drug abuse information in view of the physician's notes that the physician recorded while the patient was visiting. Here, the patient's notes may describe the patient's symptoms, appearance, and behavior. The drug abuse analysis module 230 can compare the physician's notes to the drug abuse information to determine whether the patient is exhibiting signs that are likely attributed to abuse of the target drug. In various embodiments, the drug abuse analysis module 230 may deem the target drug as failing the abuse validation process if the patient is exhibiting signs that are likely due to abuse of the target drug.

In various embodiments, the drug abuse analysis module 230 further analyzes patient information accessed from the profile data store 155 to understand the medical history of the patient. Given the medical history of the patient included in the patient information, the drug abuse analysis module 230 can determine whether the patient is likely abusing the target drug. In one embodiment, drug abuse analysis module 230 determines that the patient is likely abusing the target drug based on actions performed by the patient. For example, the actions performed by the patient can indicate a refill frequency for the target drug. If the medical history of the patient indicates that the patient has refilled a prescription for the same target drug numerous times over the past month when in fact, the prescribed drug should theoretically be refilled once a month, then the drug abuse analysis module 230 can determine that the patient is likely abusing the target drug. As another example, if the medical history of the patient indicates that the patient has obtained prescriptions for the target drug from numerous physicians over a short timeframe, drug abuse analysis module 230 may determine that the patient is likely abusing the target drug and avoiding suspicion by obtaining prescriptions from different physicians. In these examples, the drug abuse analysis module 230 deems that the target drug fails the abuse validation process.

The drug dispatch module 240 provides a target drug to the patient 110. Specifically, the drug dispatch module 240 provides a target drug to the patient 110 after the target drug has successfully undergone one or more of the safety validation process, cost validation process, and abuse validation process. In particular embodiments, the drug dispatch module 240 provides a target drug to the patient 110 after the target drug has successfully passed each of the safety validation process, cost validation process, and abuse validation process. The patient 110 can receive the target drug at an address (e.g., residential address) of the patient's choice.

In various embodiments, the drug dispatch module 240 provides the target drug to the patient 110 by performing a confirmation process. In one embodiment, the drug dispatch module 240 communicates with the user kiosk 125 to perform the confirmation process. For example, the drug dispatch module 240 transmits information about the target drug, which has passed one or more of the safety validation, cost validation, and abuse validation processes, to the user kiosk 125 such that the patient can confirm delivery of the target drug. In one embodiment, the patient can confirm a delivery address, a delivery time, personal information of the patient, and the like. Therefore, once the user kiosk 125 provides a confirmation, the drug dispatch module 240 can subsequently provide the target drug to the patient 110. An example of the confirmation process shown on example user interfaces of the user kiosk 125 is described in further detail below in relation to FIGS. 4A-4E.

In one embodiment, the drug dispatch module 240 provides a target drug to the patient 110 by providing an instruction that enables the target drug to be physically sent to the patient 110. As an example, the drug dispatch module 240 can provide an instruction on a display screen that identifies the target drug, delivery information for the target drug (e.g., an address that the target drug is to be delivered to), and an indication that the target drug has passed one or more of the separate validation processes. Therefore, an individual can package the target drug in a parcel and physically send the packaged target drug to patient 110 (e.g., through the mail). In some embodiments, the drug dispatch module 240 provides a target drug to the patient 110 by dispatching the drug. For example, the centralized pharmacy 140 may physically store packaged target drugs. Therefore, the drug dispatch module 240 can cause the previously packaged target drug to be released and physically dispatched from the centralized pharmacy 140. Here, the drug dispatch module 240 can dispatch the packaged target drug without human intervention.

### Process for Providing a Validated Drug to a Patient

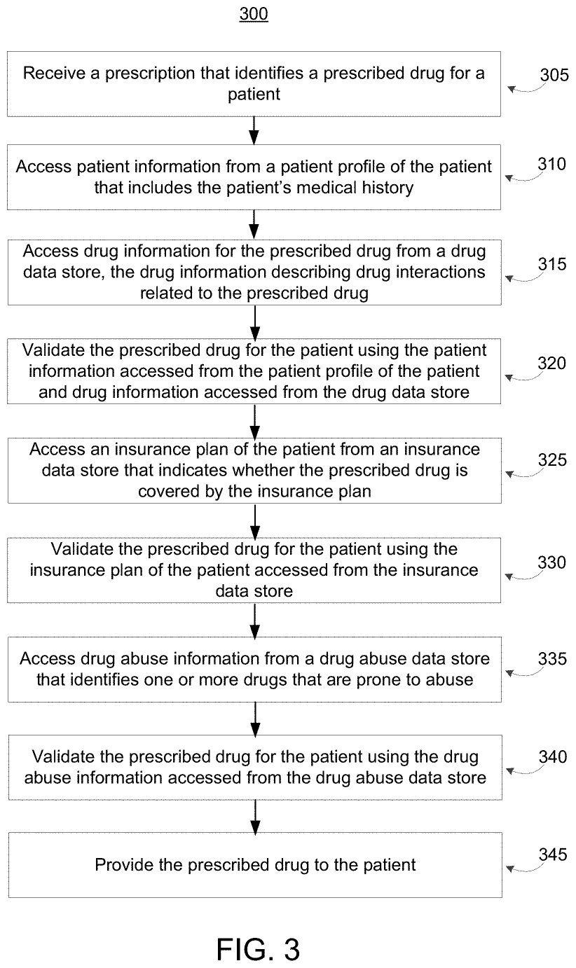

FIG. 3 depicts an example flow diagram performed by a centralized pharmacy 140 for validating and providing a drug to a patient, in accordance with an embodiment. The centralized pharmacy 140 receives 305 a prescription that identifies a prescribed drug for a patient. As an example, the prescription can be electronically provided by a physician through a physician client device 130 while the patient is visiting the physician office 120. The centralized pharmacy 140 accesses 310 a patient profile that belongs to the patient. The patient profile includes patient information, examples of which include the medical history of the patient. The centralized pharmacy 140 accesses 315 drug information from a drug data store 165. Here, the drug information can include risk information, such as possible adverse interactions, that are associated with the drug.

The centralized pharmacy 140 validates 320 the prescribed drug for the patient using the patient information accessed from the patient profile and by using the drug information accessed from the drug data store 165. As an example, the centralized pharmacy 140 validates the prescribed drug to ensure that the prescribed drug does not adversely interact with drugs identified in the patient's medical history that the patient has or is currently taking. In various embodiments, if the centralized pharmacy 140 is unable to validate the prescribed drug, the centralized pharmacy 140 identifies an alternate drug and modifies the prescription to include the alternate drug.

The centralized pharmacy 140 accesses 325 an insurance plan of the patient from an insurance data store 175. Here, the insurance plan can specify the type of health plan that the patient has purchased as well as drugs and/or treatments that are covered by the patient's health plan. The centralized pharmacy 140 validates 330 the prescribed drug for the patient using the insurance plan accessed from the insurance data store 175. In various embodiments, the centralized pharmacy 140 validates the prescribed drug by checking that the prescribed drug is covered by the patient's health plan. In some embodiments, if the centralized pharmacy 140 determines that the prescribed drug is not covered by the patient's health plan, the centralized pharmacy 140 can identify an alternate drug that is covered by the patient's health plan.

The centralized pharmacy 140 accesses 335 drug abuse information from a drug abuse data store 185 that identifies one or more drugs that are prone to abuse. The centralized pharmacy 140 can validate the prescribed drug for the patient using the information accessed from the drug abuse data store 185 and the patient information accessed from the patient's profile. Here, this validation step checks for whether the patient is potentially abusing the prescribed drug. If so, the centralized pharmacy 140 can select an alternate drug or provide an indication to the physician that the patient is likely to abuse the prescribed drug.

The centralized pharmacy 140 provides 345 the prescribed drug to the patient. For example, the centralized pharmacy 140 can physically dispatch the prescribed drug to the patient. In various embodiments, the centralized pharmacy 140 only provides a prescribed drug to the patient if the prescribed drug has passed each of the validation processes (e.g., step 320, step 330, and step 340).

### Example User Interface Displayed on a User Kiosk

FIGS. 4A-4C each depicts example user interfaces shown on a user kiosk 125, in accordance with an embodiment. In various embodiments, the information depicted on the example user interfaces of the user kiosk 125 include information that has been processed and provided by the centralized pharmacy 140. For example, the example user interfaces of the user kiosk 125 can display, to the patient, the prescribed drug that has already passed each of the safety, cost, and abuse validation processes performed by the centralized pharmacy 140. As another example, the example user interfaces of the user kiosk 125 can display, to the patient, an alternate drug (e.g., alternative to the prescribed drug). Here, the initially prescribed drug may have failed at least one of the validation processes and therefore, the alternate drug that passes the validation processes is displayed to the patient. In various embodiments, the user kiosk 125 that depicts the example user interfaces can be accessed by a patient 110 while at the physician office 120. In these embodiments, the centralized pharmacy 140 may perform each of the validation processes in real-time after receiving the prescription from the physician such that the patient 110 can be provided the relevant information on the user interfaces of the user kiosk 125 prior to the patient 110 leaving the physician office 120.

Referring to the example user interface shown in FIG. 4A, it depicts an initial user interface 405 that the patient may be greeted with when first accessing the user kiosk 125. Here, the user kiosk 125 can present selectable options to the user. For example, as shown in FIG. 4A, a first selectable option 410 can refer to a new prescription that a physician has prescribed. Thus, the patient can fill the new prescription by interacting with the user kiosk 125. Additionally, a second selectable option 415 can refer to an existing prescription. Thus, the patient can refill a prior drug by interacting with the user kiosk 125.

The user kiosk 125 may present example user interfaces 415A and 415B subsequent to receiving a selection with either the first selectable option 410 or the second selectable option 415. Each of user interfaces 415A and 415B enable the patient to enter in login information such as an email address (shown in FIG. 4A) and a password for the profile (shown in FIG. 4B). In various embodiments, the user kiosk 125 transmits the login information to the centralized pharmacy 140 such that the centralized pharmacy 140 can retrieve information describing the drugs that have been prescribed for the patient.

After the patient provides login information, the user kiosk 125 can display a user interface that depicts the patient information of the patient and drug information of drugs for the patient. In various embodiments, the user kiosk 125 receives the patient information from the centralized pharmacy 140 which has previously accessed the patient information from the patient profile data store 155 of the patient data system 150. Additionally, the user kiosk 125 can receive the drug information from the centralized pharmacy 140 which has previously accessed the drug information from the drug data store 165 of the drug data system 160. In some embodiments, the user kiosk 125 further displays the patient's insurance information. Here, the user kiosk 125 may receive the patient's insurance information from the centralized pharmacy 140 which has previously accessed the patient's insurance information from the insurance data store 175 of the insurer system 170. By displaying the patient information, drug information, and patient's insurance information, the user kiosk 125 enables the patient to view drugs of a new prescription or drugs of an existing prescription that a physician has prescribed to the patient and to provide or verify personal information of the patient (e.g., delivery address, contact phone number, etc.).

An example user interface 425 displayed by the user kiosk 125 is shown in FIG. 4D. Here, the user interface 425 displays patient information such as the physician's name 430, the patient's name 435, the patient's delivery address 475A, the preferred delivery time 475B, and the patient's phone number 475C. Additionally, the user interface 425 can display drug information such as the name 440 of each drug, the drug dose 450A, and number of available refills 450B. The user interface 425 can further display the patient's insurance information 480 as well as the cost 460 (e.g., co-pay) of each prescribed drug.

As shown in FIG. 4D, the user interface 425 may be split into two individual user interfaces 425A and 425B. For example, the first user interface 425A can be a scrollable user interface that includes individual sections 465A, 465B, 465C that each correspond to a particular drug 440. Scrolling upward or downward on the first user interface 425A can display additional sections 465 that each describe an additional drug. The second user interface 425B can display the patient information 475 that the patient can enter, edit, or verify. The second user interface 425B can further include a selectable button 470 that enables the patient to checkout and confirm the drugs that are to be dispatched to the patient. In response to the patient interacting with the selectable button 470, the user kiosk 125 can send a confirmation to the centralized pharmacy 140, and more specifically, to the drug dispatch module 240. Therefore, the user kiosk 125 can inform the drug dispatch module 240 that the patient has confirmed the information shown in the user interfaces and is expected to receive the drugs that are to be dispatched.

The user kiosk 125 may display a user interface that confirms that the drugs are to be dispatched to the patient's mailing address. As an example, the user interface 485 shown in FIG. 4E depicts a confirmation of the estimated delivery time of the drugs at the patient's mailing address. Here, the user interface 485 can include a log out selection 490 that, when selected, causes the user kiosk 125 to return to an initial user interface, such as user interface 405 shown in FIG. 4A.

### Example Computing Device

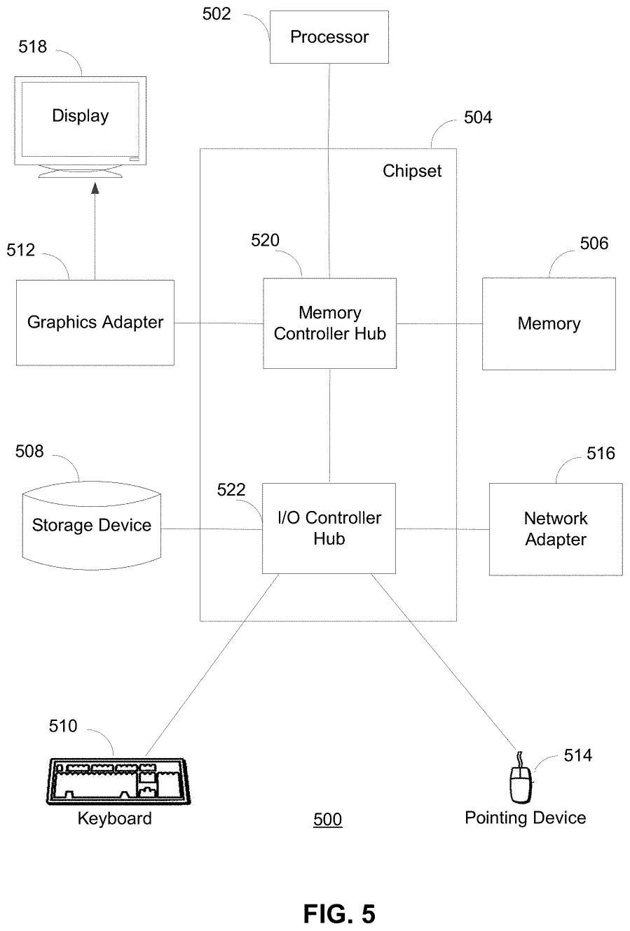

FIG. 5 depicts hardware components of an example computing device 500, in accordance with an embodiment. The example computing device 500 may be implemented in the context of the overall system environment 100 as discussed above in relation to FIG. 1. For example, the computing device 500 can be an example of the physician client device 130 and/or a device of the user kiosk 125. As another example, the computing device 500 may be the underlying hardware that performs the operations associated with the centralized pharmacy 140.

The computing device 500 includes at least one processor 502 coupled to a chipset 504. The chipset 504 includes a memory controller hub 520 and an input/output (I/O) controller hub 522. A memory 506 and a graphics adapter 512 are coupled to the memory controller hub 520, and a display 518 is coupled to the graphics adapter 512. A storage device 508, an input interface 514, and network adapter 516 are coupled to the I/O controller hub 522. Other embodiments of the computing device 500 have different architectures.

The storage device 508 is a non-transitory computer-readable storage medium such as a hard drive, compact disk read-only memory (CD-ROM), DVD, or a solid-state memory device. The memory 506 holds instructions and data used by the processor 502. The input interface 514 is a touch-screen interface, a mouse, track ball, or other type of pointing device, a keyboard, or some combination thereof, and is used to input data into the computing device 500. In some embodiments, the computing device 500 may be configured to receive input (e.g., commands) from the input interface 514 via gestures from the user. The graphics adapter 512 displays images and other information on the display 518. The network adapter 516 couples the computing device 500 to one or more computer networks.

The computing device 500 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 508, loaded into the memory 506, and executed by the processor 502.

The types of computing devices 500 can vary depending upon the embodiment and the processing power required by the entity. For example, the modules (e.g., modules 210, 220, 230, and 240) of the centralized pharmacy 140 can run in a single computing device 500 or multiple computing devices 500 communicating with each other through a network such as in a server farm. In various embodiments, the computing devices 500 lacks some of the components described above, such as graphics adapters 512, and displays 518.

### Additional Considerations

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure.

Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all of the steps, operations, or processes described.

Embodiments of the invention may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments of the invention may also relate to a product that is produced by a computing process described herein. Such a product may comprise information resulting from a computing process, where the information is stored on a non-transitory, tangible computer readable storage medium and may include any embodiment of a computer program product or other data combination described herein.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

What is claimed is:

1. A method for providing a prescription drug to a patient, the method comprising:
receiving a prescription identifying a prescribed drug for a patient;
accessing patient information from a patient profile of the patient, the patient profile including a medical history of the patient;
accessing drug information for the prescribed drug from a drug data store describing at least drug interactions related to the prescribed drug;
performing a safety validation of the prescribed drug for the patient based on the patient information accessed from the patient profile of the patient and drug information accessed from the drug data store;
accessing an insurance plan of the patient from an insurance data store, the insurance plan including at least an indication as to whether the prescribed drug is covered by the insurance plan;
performing a cost validation of the prescribed drug for the patient based on the insurance plan accessed from the insurance data store; and
based on the safety validation and the cost validation, providing the prescribed drug to the patient.

2. The method of claim 1, wherein providing the prescribed drug to the patient further comprises:
providing, for display on a user kiosk to the patient, the prescription identifying the prescribed drug for the patient;
receiving a confirmation from the user kiosk, the confirmation including an address of delivery; and
providing the prescribed drug to the address of delivery included in the confirmation.

3. The method of claim 1, wherein performing the safety validation comprises:
comparing the patient information of the patient to each of a plurality of subcategories of the drug information for the prescribed drug, the plurality of subcategories comprising drug treatment information of the prescribed drug, risk information of the prescribed drug, and relevant additional drug information.

4. The method of claim 1, wherein in response to the prescribed drug failing the safety validation, providing a notification for display on a client device, the notification identifying that the prescribed drug failed the safety validation.

5. The method of claim 1, wherein performing the cost validation comprises:
determining whether the accessed insurance plan of the patient covers the prescribed drug; and
determining whether the prescribed drug satisfies restrictions placed by an insurer, the restrictions comprising a quantity limit for the prescribed drug.

6. The method of claim 1, further comprising:
prior to providing the prescribed drug to the patient, accessing drug abuse information from a drug abuse data store, the drug abuse information identifying that the prescribed drug is prone to abuse; and
performing a third validation of the prescribed drug for the patient based on drug abuse information accessed from the drug abuse data store,
wherein providing the prescribed drug to the patient is further based on the third validation.

7. The method of claim 7, wherein the third validation is further performed based on the patient information accessed from the patient profile of the patient.

8. A method for providing a prescription drug to a patient, the method comprising:
receiving a prescription identifying a prescribed drug for a patient;
accessing patient information from a patient profile of the patient, the patient profile including a medical history of the patient;
accessing drug information for the prescribed drug from a drug data store describing at least drug interactions related to the prescribed drug;
performing a safety validation of the prescribed drug for the patient based on the patient information accessed from the patient profile of the patient and drug information accessed from the drug data store;
accessing an insurance plan of the patient from an insurance data store, the insurance plan including at least an indication as to whether the prescribed drug is covered by the insurance plan;
performing a cost validation of the prescribed drug for the patient based on the insurance plan accessed from the insurance data store; and
based on the safety validation and cost second validation, providing an instruction indicating that the prescribed drug is to be dispatched to the patient.

9. The method of claim 8, wherein providing the instruction indicating that the prescribed drug is to be dispatched to the patient further comprises:
providing, for display on a user kiosk to the patient, the prescription identifying the prescribed drug for the patient; and
receiving a confirmation from the user kiosk, the confirmation including an address of delivery,
wherein the provided instruction includes the address of delivery included in the confirmation.

10. The method of claim 8, wherein performing the safety validation comprises:
comparing the patient information of the patient to each of a plurality of subcategories of the drug information for the prescribed drug, the plurality of subcategories comprising drug treatment information of the prescribed drug, risk information of the prescribed drug, and relevant additional drug information.

11. The method of claim 8, wherein in response to the prescribed drug failing the safety validation, providing a notification for display on a client device, the notification identifying that the prescribed drug failed the safety validation.

12. The method of claim 8, wherein performing the cost validation comprises:
determining whether the accessed insurance plan of the patient covers the prescribed drug; and
determining whether the prescribed drug satisfies restrictions placed by an insurer, the restrictions comprising a quantity limit for the prescribed drug.

13. The method of claim 8, further comprising:
prior to providing the prescribed drug to the patient, accessing drug abuse information from a drug abuse data store, the drug abuse information identifying that the prescribed drug is prone to abuse; and
performing a third validation of the prescribed drug for the patient based on drug abuse information accessed from the drug abuse data store,
wherein providing the prescribed drug to the patient is further based on the third validation.

14. The method of claim 13, wherein the third validation is further performed based on the patient information accessed from the patient profile of the patient.

15. A method for providing a prescription drug to a patient, the method comprising:
receiving a prescription identifying a prescribed drug for a patient;
accessing patient information from a patient profile of the patient, the patient profile including a medical history of the patient;
accessing drug information for the prescribed drug from a drug data store describing at least drug interactions related to the prescribed drug;
performing a safety validation of the prescribed drug for the patient based on the patient information accessed from the patient profile of the patient and drug information accessed from the drug data store;
responsive to a failure of the validation of the prescribed drug, modifying the prescription by replacing the prescribed drug with an alternate drug representing an alternative to the prescribed drug;
accessing an insurance plan of the patient from an insurance data store, the insurance plan including at least an indication as to whether the alternate drug is covered by the insurance plan;
performing a cost validation of the alternate drug for the patient based on the insurance plan accessed from the insurance data store; and
based on the safety validation and the cost validation, providing an instruction indicating that the prescribed drug is to be dispatched to the patient.

16. The method of claim 15, wherein providing the instruction indicating that the prescribed drug is to be dispatched to the patient further comprises:
providing, for display on a user kiosk to the patient, the prescription identifying the prescribed drug for the patient; and
receiving a confirmation from the user kiosk, the confirmation including an address of delivery,
wherein the provided instruction includes the address of delivery included in the confirmation.

17. The method of claim 15, wherein performing the safety validation of the prescribed drug for the patient comprises:
comparing the patient information of the patient to each of a plurality of subcategories of the drug information for the prescribed drug, the plurality of subcategories comprising drug treatment information of the prescribed drug, risk information of the prescribed drug, and relevant additional drug information.

18. The method of claim 15, wherein modifying the prescription by replacing the prescribed drug with an alternate drug comprises:
identifying the alternate drug included in a subcategory of the drug information for the prescribed drug, the alternate drug having a different mode of action in comparison to the prescribed drug; and
performing a safety validation of the alternate drug to ensure that the alternate drug can be safely taken by the patient,
wherein the prescription is modified in response to the alternate drug passing the safety validation.

19. The method of claim 15, wherein performing the cost validation of the alternate drug comprises:
determining whether the accessed insurance plan of the patient covers the alternate drug; and
determining whether the alternate drug satisfies restrictions placed by an insurer, the restrictions comprising a quantity limit for the alternate drug.

20. The method of claim 15, further comprising:
prior to providing the alternate drug to the patient, accessing drug abuse information from a drug abuse data store, the drug abuse information identifying that the alternate drug that is prone to abuse; and
performing a third validation of the alternate drug for the patient based on drug abuse information accessed from the drug abuse data store,
wherein providing the alternate drug to the patient is further based on the third validation.
